SEQUENCE LISTING (2 sequences)

SEQ ID NO: 1 - RNA, 22 nt, Homo sapiens
  misc_RNA (1)..(22): hsa-miR-374a mature sequence
uuauaauacaaccugauaagug

SEQ ID NO: 2 - RNA, 72 nt, Homo sapiens
  misc_RNA (1)..(72): hsa-miR-374a stem loop
uacaucggccauuauaauacaaccugauaaguguuauagcacuuaucagauuguauuguaauugucugugua

(19) United States
(12) Patent Application Publication
(10) Pub. No.: US 2016/0348175 A1
(43) Pub. Date: Dec. 1, 2016

(54) A METHOD OF IDENTIFYING A NEONATE AT RISK OF HAVING OR DEVELOPING HYPOXIC-ISCHAEMIC ENCEPHALOPATHY (HIE)

(71) Applicant: UNIVERSITY COLLEGE CORK - NATIONAL UNIVERSITY OF IRELAND, CORK, Cork (IE)

(72) Inventors: Deirdre Murray, Cork (IE); Ann Marie Looney, Cork (IE); Geraldine Boylan, Cork (IE); John Cryan, Cork (IE)

(73) Assignee: UNIVERSITY COLLEGE CORK - NATIONAL UNIVERSITY OF IRELAND, CORK, Cork (IE)

(21) Appl. No.: 15/112,036

(22) PCT Filed: Jan. 16, 2015
(86) PCT No.: PCT/EP2015/050811

§ 371 Date: Jul. 15, 2016

Related U.S. Application Data
(60) Provisional application No. 61/928,287, filed on Jan. 16, 2014.

(30) Foreign Application Priority Data
Jan. 16, 2014 (EP) 14151458.8

(51) Int. Cl. C12Q 1/68; A61F 7/00

(52) U.S. Cl. CPC C12Q 1/6883; A61F 7/00; C12Q 2600/178

(57) ABSTRACT

A method for screening a distressed neonate for risk of having or developing HIE comprises the steps of assaying a biological sample obtained from the distressed neonate, the mother of the neonate, or from the umbilical cord or placenta, for an abundance of miR-374a in the sample, and comparing the abundance of miR-374a in the sample with a reference abundance of miR-374a, wherein a reduced abundance of miR-374a in the sample compared with the reference abundance of miR-374a is indicative of the distressed neonate being at risk of having or developing HIE. Risk of severe HIE can be determined by assaying a biological sample from the distressed neonate identified as being at risk of HIE for an abundance of a plurality of metabolites including succinate, glycerol, acetone and 3-hydroxybutyrate, providing the sum of glycerol and succinate abundance and the sum of acetone and 3-hydroxybutyrate; and correlating the sums with risk of severe HIE.

### INTRODUCTION

Neonatal hypoxic-ischaemic encephalopathy (HIE) describes the brain insult which results from insufficient oxygen or blood supply to the newborn brain during labour and delivery. HIE remains one of the leading causes of neurological disability worldwide. One in 50 newborn babies show signs of distress due to perinatal asphyxia (PA) following delivery. They may not cry, or breathe at birth, and will require immediate resuscitation. Of these 20% (4 per 1000) will go on to have hypoxic-ischaemic encephalopathy (HIE), with brain swelling, coma and seizures. In moderate and severe grades of HIE, over 50% of infants will be left with lasting brain injury, leading to cerebral palsy, learning difficulties, autism, epilepsy, visual or hearing impairment. This risk can be reduced significantly if infants are identified early and treated with induced hypothermia. This therapy has been shown to significantly improve the infant's chance of a normal outcome. However, to be beneficial it must be commenced within 6 hours of birth.

The problem to the clinicians looking after these infants is that we cannot accurately identify those babies who will benefit from hypothermia. Current blood markers (pH and lactate levels) are unreliable, but are measured in almost all infants who require resuscitation for fetal distress. EEG is currently our best method of predicting outcome. However this is difficult to record and interpret. It is also not currently available in a format which can be used in the labour ward. An early, reliable, quantifiable blood biomarker is badly needed.

It is an object of the invention to overcome at least one of the above-referenced problems.

### STATEMENTS OF INVENTION

The Applicant has discovered a micro RNA molecule, micro RNA 374a (hereafter “miR-374a”), that is expressed in reduced abundance in a distressed neonate that has or is at risk of developing severe or moderate HIE compared with a distressed neonate that did not develop HIE. This is shown in FIG. 1 and Table 1. In the cohorts tested, the mean RQ value for the control infants was 0.99153, whereas the mean RQ values for the HIE infants was 0.29463 and for the Asphyxia infants was 0.62270. Thus, miR-374a levels in neonate or maternal blood can be used to differentiate infants with, or at risk of developing, HIE from normal infants and infants with perinatal asphyxia. In particular, the invention may be employed to screen distressed neonates for risk of having or developing HIE, particularly moderate or severe HIE.

Accordingly, the invention provides a method of identifying risk of HIE in a neonate, typically a distressed neonate, comprising a step of assaying a biological sample obtained from the neonate, mother of the neonate, or placenta or umbilical cord for an abundance of miR-374a in the sample, wherein a reduced abundance of miR-374a in the sample relative to a reference abundance of miR-374a is indicative of a risk of HIE in the neonate.

In one embodiment, the invention provides a method of identifying risk of HIE in a distressed neonate comprising a step of assaying a biological sample obtained from the distressed neonate, mother of the distressed neonate, or placenta or umbilical cord within 6 hours of birth for an RQ-value of miR-374a in the sample, wherein an RQ-value of less than 0.6 is indicative of a risk of HIE in the neonate.

In another embodiment, the invention provides a method of identifying risk of moderate or severe HIE in a distressed neonate comprising a step of assaying a biological sample obtained from the distressed neonate, mother of the distressed neonate, or placenta or umbilical cord within 6 hours of birth for an RQ-value of miR-374a in the sample, wherein an RQ-value of less than 0.3 is indicative of a risk of moderate or severe HIE in the neonate.

In a further aspect, the invention provides a method of identifying risk of perinatal asphyxia in a neonate, comprising a step of assaying a biological sample obtained from the neonate, mother of the neonate, or placenta or umbilical cord prior to delivery for an abundance of miR-374a in the sample, wherein a reduced abundance of miR-374a in the sample relative to a pre-natal reference abundance of miR-374a is indicative of a risk of perinatal asphyxia in the neonate.

In one embodiment, the invention provides a method of identifying risk of severe HIE in a neonate, comprising the steps of:

(a) identifying a neonate at risk of HIE in a neonate (ideally a distressed neonate) according to a method of the invention; and
(b) assaying a biological sample from the neonate predicted as being at risk of HIE according to step (a) for an abundance of a plurality of metabolites including succinate, glycerol, acetone and 3-hydroxybutyrate, providing (i) the sum of glycerol and succinate abundance and (ii) the sum of acetone and 3-hydroxybutyrate, and correlating the sums with risk of severe HIE.

For example, when (i) the sum of glycerol and succinate abundance is greater than (ii) the sum of acetone and 3-hydroxybutyrate, this would indicate risk of severe HIE, or when (i) the sum of glycerol and succinate abundance is less than (ii) the sum of acetone and 3-hydroxybutyrate, this would indicate low risk of severe HIE.

In another embodiment, the invention provides a method of predicting severe outcome due to HIE in a neonate, comprising the steps of:

(a) identifying a neonate at risk of HIE according to a method of the invention; and
(b) assaying a biological sample from the neonate identified as being at risk of HIE according to step (a) for an abundance of a plurality of metabolites including succinate, glycerol, acetone and 3-hydroxybutyrate, providing (i) the sum of glycerol and succinate abundance and (ii) the sum of acetone and 3-hydroxybutyrate, and correlating the sums with risk of severe outcome due to HIE.

For example, when (i) the sum of glycerol and succinate abundance is greater than (ii) the sum of acetone and 3-hydroxybutyrate, this would indicate risk of severe outcome due to HIE, or when (i) the sum of glycerol and succinate abundance is less than (ii) the sum of acetone and 3-hydroxybutyrate, this would indicate low risk of severe outcome due to HIE.

In another aspect, the invention provides a method of predicting risk of severe HIE in a neonate, comprising the steps of assaying a biological sample from the neonate for an abundance of a plurality of metabolites including succinate, glycerol, acetone and 3-hydroxybutyrate, providing (i) the sum of glycerol and succinate abundance and (ii) the sum of acetone and 3-hydroxybutyrate, and correlating the sums with risk of severe HIE.

In another aspect, the invention provides a method of predicting risk of severe outcome due to HIE in a neonate, comprising the steps of assaying a biological sample from the neonate for an abundance of a plurality of metabolites including succinate, glycerol, acetone and 3-hydroxybutyrate, providing (i) the sum of glycerol and succinate abundance and (ii) the sum of acetone and 3-hydroxybutyrate, and correlating the sums with risk of severe outcome due to HIE.

In one embodiment, the correlation step comprises providing a function of (i) the sum of glycerol and succinate abundance and (ii) the sum of acetone and 3-hydroxybutyrate, and correlating the function with risk of severe HIE or severe outcome due to HIE.

The function may be (i) the sum of glycerol and succinate abundance divided by (ii) the sum of acetone and 3-hydroxybutyrate, in which case a number greater than 1 correlates with risk of severe HIE or sever outcome (i.e. 1.3) and a number less than one correlates with low risk of HIE or severe outcome (i.e. 0.33). The function may also be a ratio of (i) the sum of glycerol and succinate abundance and (ii) the sum of acetone and 3-hydroxybutyrate, in which case a higher ratio correlates with risk of severe HIE or severe outcome (i.e. a ratio greater than 1:1, for example 1.6:1) and a lower ratio correlates with low risk of severe HIE or severe outcome (i.e. a ratio less than 1:1, for example 1:3). The function may also be (i) the sum of glycerol and succinate abundance minus (ii) the sum of acetone and 3-hydroxybutyrate, in which case a positive number correlates with risk of severe HIE or severe outcome (i.e. 23) and a negative number correlates with low risk of severe HIE or sever outcome (i.e. −107).

It will be appreciated that the same sample may be used to determine the abundance of miR-374a and the metabolites, and that the assay may be run sequentially, or in tandem. In one embodiment, an initial assessment based on miR-374a levels is carried out to determine risk of HIE, and if risk of HIE is detected, then infants identified as being at risk can then be screened for risk of severe HIE (or severe outcome) using the metabolite-based method of the invention.

The invention provides a method of treating a neonate at risk of having HIE (or severe HIE), the method comprising the steps of:

(a) identifying a neonate at risk of having HIE (or severe HIE) according to a method of the invention, and
(b) treating the neonate identified as being at risk of having HIE (or severe HIE) in step (a) with a neuroprotective therapy.

The invention provides a method of treating a neonate identified as having a risk of having HIE (or severe HIE) according to a method of the invention, the method comprising the steps of treating the neonate identified as being at risk of having HIE (or severe HIE) with a neuroprotective therapy.

The invention also provides a system for determining whether a neonate is at risk of having or developing HIE, the system comprising:
(a) a determination module configured to receive at least one test sample (i.e. serum or whole blood) and perform at least one test analysis on the test sample to detect the abundance of miR-374a,
(b) optionally, a storage system for storing data relating to the abundance of the miR molecules generated by the determination system;
(c) a comparison module for comparing the detected abundance of the miR-374a with a reference abundance for the same micro RNA molecule
(d) a display module for displaying a content based in part on the data output from said determination module, wherein the content comprises a signal indicative of the presence or absence of decreased abundance of miR-374a relative to the reference abundance.

### DEFINITIONS

In this specification, the term “hypoxic-ischaemic encephalopathy” or “HIE” describes the brain insult which results from insufficient oxygen or blood supply to the newborn brain during labour and delivery, and consequent brain pathology, brain swelling or later brain injury. As employed herein, the term HIE should be understood to encompass mild, moderate or severe HIE. Moderate and severe HIE are characterised by lethargy, hypotonia, diminished deep tendon reflexes, occasional periods of apnoea, seizures, and absence of grasping, Moro, and sucking reflexes. Typically, the term HIE should be understood to include neonatal encephalopathy.

In most cases, the invention is directed to a method of screening a distressed neonate for risk of having or developing HIE, especially moderate or severe HIE. Preferably, the reference abundance of miR-374a is from a distressed neonate with perinatal asphyxia. In this specification, the term “distressed neonate” should be understood to mean a neonate that exhibits signs of fetal distress and requires resuscitation at birth. This can be determined by an attending clinician or midwife. However, in certain embodiments, the invention may be employed to predict perinatal asphyxia, in which case the biological sample is assayed for miR-374 abundance, wherein detection of a reduced abundance of miR-374a in the sample relative to a healthy control reference abundance of miR-374a is indicative of a risk of perinatal asphyxia in the neonate. In certain embodiments, the invention may be employed to predict perinatal asphyxia prior to delivery, in which case the biological sample is taken during labour (for example, a sample of maternal blood).

In this specification, the term “micro RNA-374a” or “miR-374a” should be understood to mean the micro RNA described in the miRBase database under Accession number MI0000782 (WWW.MIRBASE.ORG). The sequence of miR-374a (mature), and miR374a (stem loop), are provided below as SEQUENCE ID NO'S 1 and 2, respectively.

(SEQUENCE ID NO: 1)
UUAUAAUACAACCUGAUAAGUG
(SEQUENCE ID NO: 2)
UACAUCGGCCAUUAUAAUACAACCUGAUAAGUGUUAUAGCACUUAUCAG
AUUGUAUUGUAAUUGUCUGUGUA

In this specification, the term “neonate” should be understood to mean an infant mammal, typically an infant human, in the first 28 days after birth.

The term “at risk of having or developing HIE” refers to a risk that is greater than the 4 per 1000 risk that every neonate has of developing HIE following delivery. When the neonate being assessed is one that demonstrates signs of distress following delivery, the term “at risk of having or developing HIE” should be understood to mean a risk that is greater than the 1 per 5 risk that distressed neonates have of developing HIE following delivery.

In this specification, the biological sample from the neonate or mother should be understood to mean any biological sample including biological fluids such as blood, or a sample of cells or tissue, such as a sample of skin cells from the neonates scalp. The biological sample from the placenta or umbilical cord may be tissue, cells or blood, and the blood from the placenta or cord may be of venous or arterial origin. The term “blood” as used herein should be understood to mean blood or a blood derivative, for example plasma or serum. Generally, the biological sample is obtained postnatally, although in some circumstances it may be obtained prenatally, for example within 90 or 60 minutes before delivery. In most cases, the sample will be obtained post-natally, and generally within 6, 5, 4, 3, 2 or 1 hours of birth.

In this specification, the term “assaying the sample for an abundance of miR-374a in the sample” should be understood to mean determining the abundance of the micro RNA in either an absolute or relative manner. For example, in one embodiment of the invention, the abundance of miR-374a is determined is a relative manner using RQ-PCR. In this technique, the RQ stands for relative quantification and is generally measured as a delta-delta CT value. Essentially the abundance of a house keeping gene (for example, mir-223, however other housekeeping genes may be employed and will be known to those skilled in the art) and the target gene (mir-374a) is measured in the sample and the sample abundance is normalised according to that housekeeping gene. As the abundance of the housekeeping gene remains constant between all samples, the RQ value describes the change in expression of the target in relation to the housekeeping gene. In one embodiment, and using this technique, an RQ value of less than 0.6 is indicative of risk of HIE in the infant. The risk of HIE increases as the RQ value decreases, so that RQ values of less than 0.3 or 0.2 indicate a strong risk of HIE in the neonate, and in particular a strong risk of moderate or severe HIE.

In another embodiment, miR-374a can be determined in an absolute manner, using RT-PCR, and the absolute value correlated with risk by comparison with a reference value. Generally with this technique, the absolute value is normalised with a housekeeping gene to give a normalised mRNA copy number value, and it is this value that is compared with a normalised copy number value from a control sample. In one embodiment, the reference value from the healthy control may be a reference value from a pool of healthy controls, thus giving a mean healthy control value (or mean normalised healthy control value). The reference value may also be from a neonate with perinatal asphyxia who does not have and did not develop HIE, or a pool of such neonates.

Preferably the biological sample is assayed for abundance (relative or absolute) of miR-374a using a point-of-care device. Examples of suitable point-of-care devices capable of relative or absolute quantification of micro RNA in biological samples are known to a person skilled in the art and are described in Vaca et al (Sensors (Basel) May 2014; 14(5): 9117-9131), Liong et al (Adv. Healthcare Mater. 2014 DOI: 10.1002/adhm.201300672), and Khan et al (Anal. Chem. 2011, 83, 6196-6201).

In this specification, the term “reduced abundance of miR-374a” should be understood to mean an abundance of miR-374a that is significantly reduced compared with a reference abundance of miR-374a.

The term “reference abundance of miR-374a” should be understood to mean an abundance of miR-374a in a distressed neonate that did not develop HIE—the reference value may be obtained from a single neonate, or more preferably is obtained from a cohort of neonates, so that the reference value represents a mean value for a sample of distressed neonates that did not develop HIE. When abundance of miR-374a is determined relatively using RQ values, typically an RQ value of less than 0.62 is indicative of risk of HIE, where the risk of HIE increases as the RQ value decreases (for example, RQ values of less than 0.5, 0.4, 0.3, 0.2 or 0.1). In one embodiment of the invention, an RQ value of less than 0.3 is indicative of risk of severe or moderate HIE, where the risk of severe or moderate HIE increases as the RQ value decreases (for example, RQ values of less than 0.2 or 0.1).

In embodiments of the invention directed to screening for risk of perinatal asphyxia (either pre- or post-delivery), the reference level of miR-374a (described herein as “healthy control reference abundance of miR-374a”) should be understood to mean an abundance of miR-374a in one or more healthy neonates exhibiting no signs of distress or perinatal asphyxia.

In this specification, the term “severe outcome due to HIE” should be understood to mean death prior to 3 years of age, severe cerebral palsy, or a composite score <70, i.e. 2 standard deviations below the mean (mean+100, SD+15) in all 3 of the cognitive, language and motor subscales of the Bayley Scales of Infant and Toddler Development Edition III (BSIDIII).

In this specification, the term “function of the (i) the sum of glycerol and succinate abundance and (ii) the sum of acetone and 3-hydroxybutyrate” may be any one of a number of functions, including: (A) (i) the sum of glycerol and succinate abundance divided by (ii) the sum of acetone and 3-hydroxybutyrate, in which case a number greater than 1 correlates with risk of severe HIE (i.e. 1.3) and a number less than one correlates with low risk of HIE (i.e. 0.33); (B) a ratio of (i) the sum of glycerol and succinate abundance and (ii) the sum of acetone and 3-hydroxybutyrate, in which case a higher ratio correlates with risk of HIE (i.e. a ratio greater than 1:1, for example 1.6:1) and a lower ratio correlates with low risk of HIE (i.e. a ratio less than 1:1, for example 1:3); and (C) (i) the sum of glycerol and succinate abundance minus (ii) the sum of acetone and 3-hydroxybutyrate, in which case a positive number correlates with risk of HIE (i.e. 23) and a negative number correlates with low risk of HIE (i.e. −107). Other functions capable of discriminating severe HIE from moderate and mild HIE (or from control or asphyxia patients) may be employed and will be apparent to a person skilled in the art, for example (i) the log of the sum of glycerol and succinate abundance divided by (ii) the log of the sum of acetone and 3-hydroxybutyrate.

In this specification, the term “neuroprotective therapy” should be understood to mean a treatment for neonatal hypoxic-ischemic brain injury, including HIE, and includes treatment with therapeutic hypothermia, Xenon gas inhalation, stem cell transplantation, mast cell stabilisers, allopurinol, melatonin or any other neuroprotective agents or therapies. Stem cell therapy for neonatal HIE is described in Gonzales-Portillo et al (Frontiers in Neurology, August 2014, Vol. 5, Article 147). Xenon therapy is described in Azzopardi et al (Arch. Dis. Child Fetal Neonatal Ed. 2013; 98 F437-F439). Neuroprotective agents for neonates are described in Robertson et al (The Journal of Pediatrics Vol. 160, No: 4) and include Tetrahydrobiopterin (FDA approved), Melatonin (FDA approved), nNOS inhibitors, Xenon gas, Allopurinol (FDA approved), Vitamins C and E (FDA approved), N-acetylcysteine (FDA approved), Erythropoietin (FDA approved), and Epo mimetics.

The methods and systems for detecting risk of HIE as described herein are based on measuring the abundance of miR-374a in a distressed neonate and comparing the measured level with a reference level in one or more distressed neonates that did not develop HIE, in which reduced abundance relative to the reference level is indicative of risk of HIE. However, it will be appreciated that the reference level may be from a neonate with HIE, in which case a measured level that is similar to the reference level will indicate a risk of HIE.

The invention also provides a system for obtaining data from at least one test sample obtained from at least one individual, the system comprising:
(a) a determination module configured to receive at least one test sample (i.e. serum or whole blood) and perform at least one test analysis on the test sample to detect decreased abundance of miR-374a relative to a reference abundance,
(b) optionally, a storage system for storing data relating to the abundance of the miR molecules generated by the determination system; and
(c) a display module for displaying a content based in part on the data output from said determination module, wherein the content comprises a signal indicative of the presence or absence of decreased abundance of miR-374a.

Preferably, the determination system comprises means for measuring the level of a micro RNA molecule (in absolute or relative terms) and then comparing the measured level with a reference level. The reference level may be a level of the same micro RNA from one or more healthy neonates, typically from the same type of sample.

Ideally, the determination system comprises a PCR apparatus.

The invention also provides a system for determining whether a neonate is at risk of having or developing HIE, the system comprising:
(e) a determination module configured to receive at least one test sample (i.e. serum or whole blood) and perform at least one test analysis on the test sample to detect the abundance of miR-374a,
(f) optionally, a storage system for storing data relating to the abundance of the miR molecules generated by the determination system;
(g) a comparison module for comparing the detected abundance of the miR-374a with a reference abundance for the same micro RNA molecule
(h) a display module for displaying a content based in part on the data output from said determination module, wherein the content comprises a signal indicative of the presence or absence of decreased abundance of miR-374a relative to the reference abundance.

In another embodiment, the system is for determining a suitable treatment for a neonate with HIE. Suitably, the comparison module is adapted to detect decreased abundance of miR-374a, and the display module is adapted to displaying a content based in part on the data output from said determination module, wherein the content comprises (a) a signal indicative of decreased abundance of miR-374a and/or (b) a content comprising a signal indicative of whether the individual is suitable for treatment with a neuroprotective therapy.

Embodiments of the invention also provide for systems (and computer readable media for causing computer systems) to perform a method for identifying the risk of a neonate, especially a distressed neonate, having HIE, or determining a suitable treatment for a neonate identified as being at risk of having HIE.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The functional modules of certain embodiments of the invention include at minimum a determination module, a storage module, optionally, a comparison module, and a display module. The functional modules can be executed on one, or multiple, computers, or by using one, or multiple, computer networks. The determination system has computer executable instructions to provide e.g., sequence information in computer readable form.

The storage module which can be any available tangible media that can be accessed by a computer. The storage module (i.e. computer readable storage media) includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable storage media may define instructions, for example, as part of one or more programs, that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable storage media on which such instructions are embodied may reside on one or more of the components of either of a system, or a computer readable storage medium described herein, may be distributed across one or more of such components.

The computer-readable storage media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The determination module can comprise any system for detecting increased or decreased abundance of the relevant micro RNA molecule. Standard procedures such as quantitative PCR can be used. Additionally one can determine other factors relevant to diagnosis of HIE, for example blood pH. These factors can be used in conjunction with levels of the relevant micro RNA molecule. The information determined in the determination system can be read by the storage device. As used herein the “storage device” is intended to include any suitable computing or processing apparatus or other device configured or adapted for storing data or information. Examples of an electronic apparatus suitable for use with the present invention include a stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon nucleic acid sequence information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising information relating to micro RNA levels.

In one embodiment the reference data stored in the storage device to be read by the comparison module is compared for the purpose of detecting increased or decreased abundance of one or more specific micro RNA molecules compared with a reference abundance.

The “comparison module” can use a variety of available software programs and formats for the comparison operative to compare micro RNA abundance determined in the determination system to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to the abundance of one or more micro RNA molecules in a sample relative to a reference abundance (i.e. relative to abundance of miR-374a in one or more healthy neonates).

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that may be stored and output as requested by a user using a display module.

In one embodiment of the invention, the content based on the comparison result is displayed on a computer monitor. In one embodiment of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The methods described herein therefore provide for systems (and computer readable media for causing computer systems) to perform methods as described above, for example (a) methods of detecting risk of a neonate, typically a distressed neonate, having HIE.

### BRIEF DESCRIPTION OF THE FIGURES

FIG. 1: Bar graph of miR-374a expression in control infants vs. perinatal asphyxia infants vs. HIE infants (n=70)

* represents a statistically significant p value of <0.05 between control and asphyxia,
** represents a statistically significant p value of <0.01 between control and HIE,
+ represents a statistically significant p value of <0.05 between asphyxia and HIE Data presented as Mean+/−SEM

FIG. 2: Glycerol+succinate/acetone+3-hydroxybutyrate and clinical grade of encephalopathy

FIG. 3: Glycerol+succinate/acetone+3-hydroxybutyrate and neurological outcome at 3 years. Severe defined as death, spastic quadriplegia or less than 70 on the Bayley Scales of Infant Development

### DETAILED DESCRIPTION OF THE INVENTION

### Material and Methods

### Patient Selection

Ethical approval for this study was obtained from the Clinical Research Ethics committee of the Cork Teaching Hospitals. The study was conducted from May 2009 to June 2011 in a single maternity hospital with 9000 deliveries per annum. Infants were identified as being at risk for HIE if they were over 36 weeks gestation with one or more of these previously published risk factors: an arterial cord pH<7.1, 5 minute Apgar score ≦6, or resuscitation at delivery required intubation. Parents of neonates meeting inclusion criteria were approached and written informed consent obtained. After enrolment clinical and demographic details on all infants were recorded prospectively. Grade of encephalopathy was assigned at 24 hours of life by a dedicated research fellow, using the modified Sarnat score. Standardised neurological assessment was additionally performed on day 3 and at discharge.

Case infants were divided into those with HIE, and those with biochemical or clinical risk of asphyxia without clinical encephalopathy (Asphyxia) based upon this examination.

A control population was recruited over the same period as part of an ongoing birth cohort study (The BASELINE Study www.baselinestudy.net). Ante-natal parental consent was obtained for all control infants enrolled. The control population were all full term infants, born by unassisted vaginal delivery, without any medical issues. All had normal examinations, were not admitted to the neonatal unit, and did not have EEG monitoring.

All case infants had continuous multi-channel EEG recorded, commencing in the first 24 hours of life. The background EEG was graded according to a modification of a standardized HIE grading system. The entire recording was reviewed for the presence of electrographic seizures, which were defined as stereotyped repetitive discharges on one or more channels, with a clear evolution, that lasted for >10 seconds.

All cases underwent EEG monitoring, as soon as possible after delivery, during the first 24 hours of life or longer as required, except one infant who passed away prior to arrival in the neonatal unit. Silver-silver chloride EEG electrodes were applied to the scalp at F3, F4, C3, C4, T3, T4, O1, O2, Cz (according to the international 10-20 system modified for neonates). The EEG was recorded on a NicOne video-EEG system (Carefusion, Madison, Wis.). The video-EEG was then reviewed by an experienced neonatal neurophysiologist (G.B.B) and analysed for background features described by Murray et al. (Murray et al., 2009), seizure burden and sleep-wake cycling. The EEG was examined as 1 hour epochs at 6 hours of life, or earliest available recorded time-point, and at 24 hours of life. An EEG grade was assigned at these time-points and designated as normal (sleep cycles present on continuous background), mildly abnormal (e.g. continuous but abnormalities of sleep cycles), moderately abnormal (e.g. discontinuity or presence of seizures) and severely abnormal (suppression/isoelectric tracing, high seizure burden/status).

Therapeutic hypothermia, whole body cooling according to the TOBY registry protocols, was commenced at the discretion of the supervising clinician on duty.

A matched control population was recruited over the same period as part of an ongoing birth cohort study (The BASELINE study www.baselinestudy.net). The controls were matched to cases for both infant and maternal demographic parameters including; gestational age, gender, birth weight, and centile, method of delivery, maternal ethnicity, maternal age, and maternal BMI. Antenatal consent was obtained for all control infants enrolled. Controls did not have any clinical signs of asphyxia, or other medical issues at delivery. Clinically they were healthy, had normal examination and did not require EEG monitoring.

### Umbilical Cord Blood Sampling and Storage:

Umbilical cord blood was drawn for all infants using identical standardised operating procedures. Six ml of mixed umbilical cord blood was drawn from the cord, and placed in a plain serum tube (BD Vacutainer no. 366431) within 20 min of placental delivery. Serum was allowed clot for 30 min at 4° C., then centrifuged (2400×g, 10 min, 4° C.). The serum was pipetted into a second spin tube, and centrifugation repeated (3000×g, 10 min, 4° C.). Clean serum was then aliquoted into lithium heparin microtubes (VWR no. 89179-704) and stored at −80° C. until analysis. Total time from birth to samples being frozen at −80° C. was always under 3 hours.

### MiRNA Analysis

### Sample Collection, and RNA Extraction

Umbilical cord blood was drawn on all infants. 3 ml of cord blood was placed into Tempus™ Blood RNA tubes (Applied Biosystems, Foster City, Calif.). The tubes were then agitated for 10 seconds to ensure that the reagent made uniform contact with the sample, before being biobanked at −80 C. Once sufficient samples were collected, the RNA was extracted from the Tempus system using the MagMAX™ for Stabilized Blood Tubes RNA Isolation Kit (Applied Biosystems/Ambion, Austin, Tex.). The concentration of the RNA was determined using a NanoDrop Spectrophotometer (Rockland, Del.).

### MiRNA Microarray Assay

For the microarray assay a commercial provider was used (Beckman Coulter Genomics Inc., Fullerton, Ca.). Beckman Coulter use the Agilent Human miRNA Microarray Version 3.0 (Agilent technologies Inc., Agilent Laboratories, Santa Clara, Ca.). This system contains probes for 866 human miRNA from the Sanger miRBase 12.0 Release (http://www.mirbase.org). In brief the system directly labels the miRNA, adding a single 3′ Cytidine and one cyanine dye to the 3′ end. When developing their probes, Agilent added a Guanine base to the 5′ end of each, this complements the 3′ Cytidine added during labeling, allowing G-C pair to form, stabilising the targeted miRNA. This stabilisation allows equalisation of the melting temperatures of the probe-target hybrids. Additionally there is a further 5′ hairpin on the probes, which increases the target specificity of the probe, preventing stable hybridization of longer non-target miRNAs.

### MiRNA Real Time PCR

Quantitative reverse transcription polymerase chain reaction (RT-PCR) was performed for hsa-mir-374a, to validate the microarray results. For this analysis the miRCURY LNA™ Universal RT microRNA PCR (Exiqon, Woburn, Ma) was used with pre designed primers (Exiqon, Woburn, Ma) for the miRNA of interest (hsa-mir-374a} and housekeeper miRNA (has-mir-223).

All analysis was performed as per the manufacturer's protocols for individual assays using whole blood samples. In brief, RT master mix was made for each of the primers as per the kits protocols. First-strand cDNA synthesis was then performed by adding 16 μL of RT master mix to 4 μL of template total RNA. This was incubated at 42° C. for 60 min, and then inactivated by heating to 95° C. for 5 min. The cDNA was then added to the PCR master mix (PCR primer and SYBR Green master mix) and centrifuged at 1500 g for 1 min to ensure all reagents were mixed. For amplification all reactions were performed in duplicate, at a final volume of 10 μL per well, using Rotor Gene 6000. Polymerase activation and denaturation was performed at 95° C. for 10 min, followed by 40 amplification cycles of 95° C. for 10 s and 60° C. for 60 s, with a ramp-rate of 1.6° C./s. At the end of the PCR cycles, melting curve analyses were performed. Threshold values for threshold cycle determination (CO were generated for each of the duplicate amplification reactions, and the mean calculated. The miRNA fold change relative to the housekeeper miRNA was then calculated using the delta-delta Ct method.

### ¹H-NMR Metabolomic Analysis:

We have previously reported a detailed description of the metabolomic method (Reinke et al., 2013). In brief, the BioVision Deproteinizing Sample Preparation Kit (Milpitas, Calif., USA) was used to remove protein. Protein was precipitated using perchloric acid and the pH of the supernatant was adjusted if necessary. Sample volume was brought to 190 μl with water. Ten μl of 5 mM 2,2-dimethyl-2-sila 3,3,4,4,5,5,-hexadeutero-pentane sulphonic acid (DSS-d₆, Chenomx Inc., Edmonton, Alberta, Canada) was added as a concentration reference and chemical shift indicator. Samples were centrifuged and the clarified serum was transferred to 3 mm NMR tubes.

One-dimensional ¹H-NMR spectra were acquired using a 600 MHz Varian Inova spectrometer with a Varian Unibody 3 mm ¹H¹⁹F probe (Varian Inc., Palo Alto, Calif., USA), and spectra were acquired using a tnnoesy pulse sequence (Vnmr 6.1B software, Varian Inc.). The 600 MHz database provided in Chenomx NMR Suite Professional software v5.1 (Chenomx Inc., Edmonton, Alberta, Canada) was used for metabolite identification and quantification of the 1D spectra. Pooled quality control samples were acquired and analysed after every twentieth sample.

### Outcome:

Outcome measurement was carried out on all eligible cases between 36 to 42 months of age. The Bayley Scales of Infant and Toddler Development Edition III (BSIDIII) was administered by a research fellow (C.A), trained in administration and scoring and blinded to the clinical background of the infants. For the purpose of this work a severely abnormal outcome was designated as death, severe cerebral palsy or a composite score <70, i.e. 2 standard deviations below the mean (mean=100, SD=15) in all 3 of the cognitive, language and motor subscales of the BSIDIII. All other outcomes were designated non-severe. All controls underwent Ages and Stages parental questionnaire at 2 years of age under the protocol of the BASELINE study.

### Statistical Analysis:

All metabolomic data was normalised by natural log transformation. The absolute values of the four metabolites of interest (glycerol, succinate, acetone and 3-hydroxybutyrate) were analysed individually and in combination against both clinical Sarnat grading of encephalopathy, and EEG grading at 6 hours of life. For metabolites, the mean (μM) concentration and 95% confidence intervals (CI) are reported by taking the exponential of the log transformed mean and CI. Demographic information is presented as mean (standard deviation), median (interquartile range) and n (percentage). The Kruskall-Wallis H test was used to measure the difference between medians, and a One-way Analysis of Variance (ANOVA) was performed to measure differences between means, with Bonferroni correction as appropriate. For comparison of outcome groups, a non-parametric Mann-Whitney post-hoc analysis was performed. The predictive ability of the metabolite ratio for a severe outcome was assessed using the area under the receiver operating characteristic (AUROC) curve. All statistical analysis was performed using IBM SPSS statistics 21.

### Results

miR-374a Analysis

The miR-374a levels for all patients are provided below in Table 1.

TABLE 1
RQ
Description statistics of all sample groups (Control, Asphyxia, HIE)

| | | | | | 95% Confidence | | | |
|---|---|---|---|---|---|---|---|---|
| | | | | | Interval for Mean | | | |
| | | | Std. | Std. | Lower | Upper | | |
| | N | Mean | Deviation | Error | Bound | Bound | Minimum | Maximum |
| Control | 18 | 0.99153 | 0.954497 | 0.224977 | 0.51687 | 1.46619 | 0.079 | 3.39 |
| Asphyxia | 32 | 0.62270 | 0.859228 | 0.151891 | 0.31292 | 0.93249 | 0.006 | 3.72 |
| HIE | 20 | 0.29463 | 0.211021 | 0.047186 | 0.19587 | 0.39339 | 0.028 | 0.88 |
| Total | 70 | 0.62381 | 0.796929 | 0.95251 | 0.43379 | 0.81383 | 0.006 | 3.72 |

### Metabolite Study Population

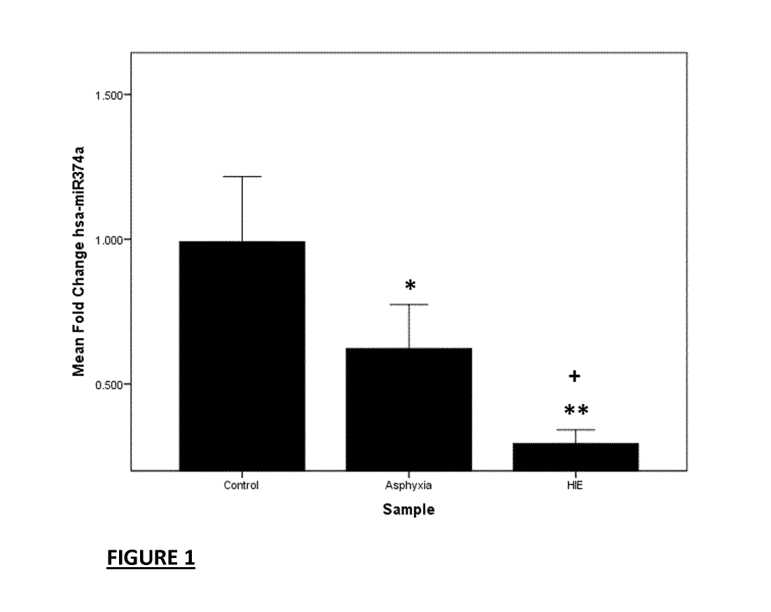

One hundred infants were recruited for the study (FIG. 1). Forty-one were excluded (15 had insufficient sample quantity for NMR analysis, 16 had no EEG, 7 had missing clinical data, 3 had alternate diagnosis), leaving 59 infant samples for metabolomic analysis. Of these 59 infants, 27 developed clinical encephalopathy according to Sarnat grading; 15 mild, 6 moderate and 6 severe and the remaining 32 were designated as perinatal asphyxia.

Of the 27 infants with clinical encephalopathy, the EEG grading at 6 hours or earliest time-point revealed 1 normal EEG (clinically mildly encephalopathic), 11 mildly abnormal (including 1 clinically moderate encephalopathy who underwent therapeutic hypothermia), 7 moderately abnormal tracings (including 4 clinically mild encephalopathies) and 8 with severely abnormal EEG findings (including 2 that were clinically moderate, one of which did not receive therapeutic hypothermia).

At 24 hours, 3 infants had normal EEGs (including 3 that were clinically mild encephalopathies), 12 had mildly abnormal EEGs (including the same clinically moderate infant mentioned above who received TH), 7 moderately abnormal (including 1 previously severely abnormal that had recovered and 1 mild encephalopathy) and 5 severely abnormal EEGs.

Outcome was determined on all severely affected infants. In the moderate group, 1 infant was lost to follow-up and 1 infant was excluded from follow-up due to alternate diagnosis. Similarly in the mild encephalopathy group, 1 infant was lost to follow-up and 1 was excluded due to alternate diagnosis. All severely abnormal outcomes (n=5) were in the severe encephalopathy group who persisted with severely abnormal EEGs at 24 hours. One infant who was, based on Sarnat score, felt to be severely encephalopathic, had actually shown EEG recovery to moderately abnormal by 24 hours of life and had a normal outcome on BSID (III) during the toddler period.

### Metabolite Analysis

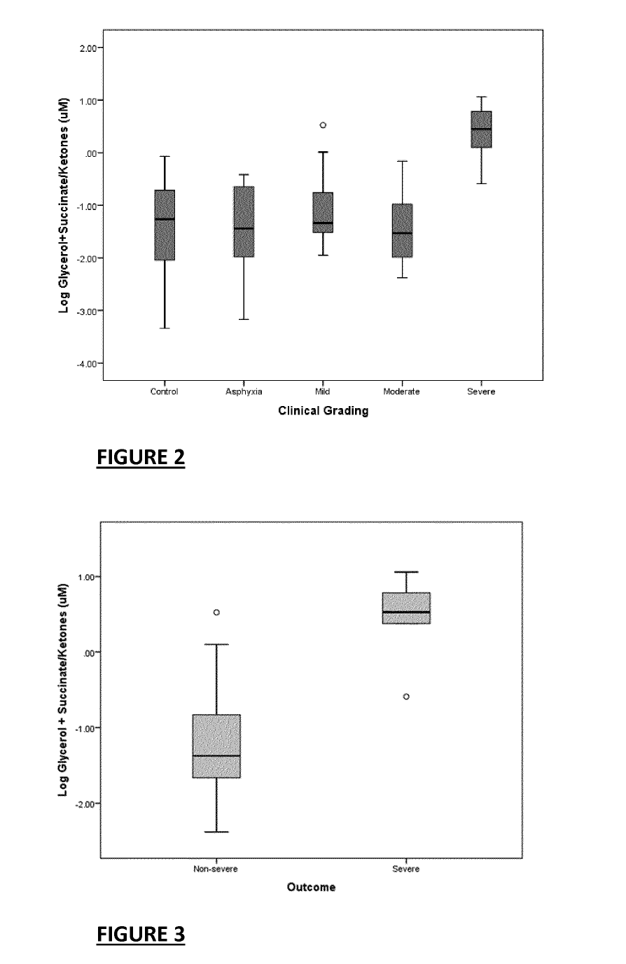

In combination the 4 metabolites ratio of glycerol+succinate/acetone+3-hydroxybutyrate retained significance in both correlation with clinical grade of encephalopathy and outcome (p=0.002 and p=0.001 respectively) FIGS. 2 and 3. This ratio also demonstrated a high predictive value for outcome with an AUC of 0.967.

The invention is not limited to the embodiments hereinbefore described which may varied in construction and detail without departing from the spirit of the invention.

1. A method of identifying risk of HIE in a distressed neonate, comprising a step of assaying a biological sample obtained from the distressed neonate, mother of the neonate, or placenta or umbilical cord, for an abundance of miR-374a in the sample, wherein a reduced abundance of miR-374a in the sample relative to a reference abundance of miR-374a is indicative of a risk of HIE in the distressed neonate.

2. The method of claim 1, wherein the biological sample is blood obtained from the umbilical cord or placenta.

3. The method of claim 1, wherein the biological sample is obtained within six hours after delivery.

4. A method of predicting risk of severe HIE or risk of severe outcome due to HIE in a neonate comprising the steps of:
(a) identifying a distressed neonate at risk of HIE, comprising a step of assaying a biological sample obtained from the distressed neonate, mother of the neonate, or placenta or umbilical cord, for an abundance of miR-374a in the sample, wherein a reduced abundance of miR-374a in the sample relative to a reference abundance of miR-374a is indicative of a risk of HIE in the distressed neonate; and
(b) assaying a biological sample from the distressed neonate identified as being at risk of HIE according to step (a) for an abundance of a plurality of metabolites including succinate, glycerol, acetone and 3-hydroxybutyrate;
(c) providing a function of the (i) the sum of glycerol and succinate abundance and (ii) the sum of acetone and 3-hydroxybutyrate; and
(d) correlating the function with risk of severe HIE, thereby predicting risk of severe HIE or risk of severe outcome due to HIE in a neonate.

5. (canceled)

6. The method of claim 4, wherein the function is selected from the group consisting of:
(A) (i) the sum of glycerol and succinate abundance divided by (ii) the sum of acetone and 3-hydroxybutyrate, in which case a number greater than 1 correlates with risk of severe HIE and a number less than one correlates with low risk of severe HIE;
(B) a ratio of (i) the sum of glycerol and succinate abundance and (ii) the sum of acetone and 3-hydroxybutyrate, in which case a higher ratio correlates with risk of severe HIE and a lower ratio correlates with low risk of severe HIE; and
(C) (i) the sum of glycerol and succinate abundance minus (ii) the sum of acetone and 3-hydroxybutyrate, in which case a positive number correlates with risk of severe HIE and a negative number correlates with low risk of severe HIE.

7. The method of claim 1, further comprising a step of treating a neonate having or at risk of having HIE
with a neuroprotective therapy.

8. The method of claim 7, wherein steps (a) and (b) are carried out within 6 hours of delivery of the neonate.

9. The method of claim 7, wherein the neuroprotective therapy is induced hypothermia.

10.-12. (canceled)

13. A method of identifying risk of perinatal asphyxia in a neonate, comprising a step of assaying a biological sample obtained from the mother, foetus, or placenta or umbilical cord prior to delivery for an abundance of miR-374a in the sample, wherein a reduced abundance of miR-374a in the sample relative to a healthy control reference abundance of miR-374a is indicative of a risk of perinatal asphyxia.

14. The method of claim 13, wherein the biological sample is obtained during labour.

15. The method of claim 13, further comprising a step of obtaining an RQ-value of miR-374a in the biological sample wherein an RQ-value of less than 0.99 is indicative of a risk of perinatal asphyxia in the neonate.

16. The method of claim 4, further comprising a step of treating a neonate predicted to have a risk of severe HIE or risk of severe outcome due to HIE with a neuroprotective therapy.
